(19) United States
(12) Patent Application Publication
(10) Pub. No.: US 2019/0038598 A1
(43) Pub. Date: Feb. 7, 2019

(54) PHARMACEUTICAL COMPOSITION INCLUDING DORZOLAMIDE AND BRIMONIDINE

(71) Applicant: Santen Pharmaceutical Co., Ltd., Osaka-shi, Osaka (JP)

(72) Inventor: Shinya UMEZAKI, Nara (JP)

(73) Assignee: Santen Pharmaceutical Co., Ltd., Osaka-shi, Osaka (JP)

(21) Appl. No.: 16/075,501

(22) PCT Filed: Feb. 21, 2017
(86) PCT No.: PCT/JP2017/006336

§ 371 Date: Aug. 3, 2018

(30) Foreign Application Priority Data
Feb. 22, 2016 (JP) 2016-031091

(51) Int. Cl. A61K 31/382; A61K 47/18; A61K 47/02; A61K 9/08; A61P 27/06

(52) U.S. Cl. CPC A61K 31/382; A61K 47/18; A61K 47/02; A61K 31/498; A61P 27/06; A61K 2300/00; A61K 9/08

(57) ABSTRACT

A pharmaceutical composition includes dorzolamide or a salt thereof and brimonidine or a salt thereof and has a pH of 6.0 or more. The pharmaceutical composition preferably does not include a preservative or includes a prescribed amount thereof, wherein said prescribed amount is preferably such that the common logarithmic value of the ratio (B/A) of the number (B) of bacteria at the time of inoculation to the number (A) of bacteria when the number of viable bacteria is measured after inoculating bacteria in a test sample that consists of the preservative and water and mixing homogeneously such that the concentration of Escherichia Coli ATCC 8739 in the bacterial suspension is within the range of 105-106 cfu/mL, and collecting 1 mL of the test sample using a micropipette after the test sample has been stored for seven days at 20-25° C. while shielded from light, is 2.0 or less.

### TECHNICAL FIELD

The present invention relates to a pharmaceutical composition that comprises dorzolamide or a salt thereof and brimonidine or a salt thereof.

### BACKGROUND ART

Dorzolamide that is a carbonic anhydrase inhibitor exhibits an effect of lowering intraocular pressure, and thus is useful for treatment of glaucoma or ocular hypertension. A formulation comprising dorzolamide is commercially available as Trusopt (registered trademark) ophthalmic solution. Moreover, a formulation comprising dorzolamide and timolol is commercially available as Cosopt (registered trademark) combined ophthalmic solution.

Furthermore, brimonidine that is an α₂ receptor agonist also exhibits an effect of lowering intraocular pressure, and thus is useful for treatment of glaucoma or ocular hypertension. A formulation comprising brimonidine is commercially available as Aiphagan (registered trademark) ophthalmic solution.

Ophthalmic solutions should have a certain or higher level of preservative efficacy in order to prevent fungi and the like from propagation associated with repeated uses of the solutions. For example, the above Trusopt (registered trademark) ophthalmic solution or Cosopt (registered trademark) combined ophthalmic solution is formulated with benzalkonium chloride as a preservative. However, benzalkonium chloride has cytotoxicity and can induce corneal epithelium disorder if exposure is increased (Non-patent document 1). Hence, benzalkonium chloride-free Cosopt (registered trademark) combined ophthalmic solution is also commercially available. This ophthalmic solution comprises no preservative, so that single-use unit-dose containers or PFMD (Preservative Free Multi Dose) containers are used for the solution.

In view of convenience, safety, production cost, and the like, a novel ophthalmic solution comprising no preservative such as benzalkonium chloride or comprising a low content thereof, or a novel ophthalmic solution that can be repeatedly used without the use of any specially structured container has been desired.

Non-Patent Document 1: The Journal of the Japan Ophthalmologists Association 58(10), 945-950(1987)

### DISCLOSURE OF THE INVENTION

### Problems to be Solved by the Invention

The present invention addresses the problem of providing a pharmaceutical composition that comprises dorzolamide or a salt thereof and brimonidine or a salt thereof and can exhibit a sufficient preservative effect in spite of comprising no preservative or comprising a low content of a preservative.

### Means for Solving the Problems

As a result of intensive studies, the present inventors have discovered that, surprisingly, a pharmaceutical composition comprising dorzolamide or a salt thereof and brimonidine or a salt thereof exhibits a sufficient preservative effect meeting the criteria “Category IA” according to The Japanese Pharmacopoeia 16th Edition (JP16) General Information “antimicrobial preservative effectiveness test” when the pH is 6.0 or more, in spite of comprising no benzalkonium chloride, and thus have completed the present invention. Specifically, the present invention provides the following (1) to (21).

(1) A pharmaceutical composition, which comprises dorzolamide or a salt thereof and brimonidine or a salt thereof, and does not comprise a preservative or comprises a preservative in a prescribed amount, wherein: the prescribed amount is such that the common logarithmic value of the ratio (B/A) of the number (B) of bacteria at the time of inoculation to the number (A) of the bacteria, when the number of the viable bacteria is measured after inoculating Escherichia Coli ATCC 8739 in a test sample that consists of the preservative and water and mixing homogeneously such that the concentration of the bacterial suspension is within the range of 10⁵-10⁶ cfu/mL, and then collecting 1 mL of the test sample using a micropipette after the test sample has been stored for seven days at 20° C.-25° C. while shielded from light, is 2.0 or less; and the pH is 6.0 or more.

(2) A pharmaceutical composition, which comprises dorzolamide or a salt thereof and brimonidine or a salt thereof, does not comprise benzalkonium chloride, and does not comprise a preservative other than benzalkonium chloride or comprises the same in a prescribed amount, wherein: the prescribed amount is such that the common logarithmic value of the ratio (B/A) of the number (B) of bacteria at the time of inoculation to the number (A) of the bacteria, when the number of the viable bacteria is measured after inoculating Escherichia Coli ATCC 8739 in a test sample that consists of the preservative and water and mixing homogeneously such that the concentration of the bacterial suspension is within the range of 10⁵-10⁶ cfu/mL, and then collecting 1 mL of the test sample using a micropipette after the test sample has been stored for seven days at 20° C.-25° C. while shielded from light, is 2.0 or less; and the pH is 6.0 or more.

(3) The pharmaceutical composition according to (1) or (2), which comprises edetic acid or a salt thereof, wherein the content of the edetic acid or a salt thereof ranges from 0.0001% (w/v) to 2% (w/v).

(4) The pharmaceutical composition according to any one of (1) to (3), which comprises boric acid or a salt thereof, wherein the content of the boric acid or a salt thereof ranges from 0.0001% (w/v) to 5% (w/v).

(5) A pharmaceutical composition, which comprises dorzolamide or a salt thereof and brimonidine or a salt thereof, does not comprise a preservative other than edetic acid or a salt thereof and boric acid or a salt thereof, or comprises the same in a prescribed amount, wherein: the prescribed amount is such that the common logarithmic value of the ratio (B/A) of the number (B) of bacteria at the time of inoculation to the number (A) of the bacteria, when the number of the viable bacteria is measured after inoculating Escherichia Coli ATCC 8739 in a test sample that consists of the preservative and water and mixing homogeneously such that the concentration of the bacterial suspension is within the range of 10⁵-10⁶ cfu/mL, and then collecting 1 mL of the test sample using a micropipette after the test sample has been stored for seven days at 20° C.-25° C. while shielded from light, is 2.0 or less; the content of the edetic acid or a salt thereof ranges from 0.0001% (w/v) to 2% (w/v); the content of the boric acid or a salt thereof ranges from 0.0001% (w/v) to 5% (w/v); and the pH is 6.0 or more.

(6) A pharmaceutical composition, which comprises dorzolamide or a salt thereof and brimonidine or a salt thereof, does not comprise a preservative other than edetic acid or a salt thereof and boric acid or a salt thereof, wherein: the content of the edetic acid or a salt thereof ranges from 0.0001% (w/v) to 2% (w/v); the content of the boric acid or a salt thereof ranges from 0.0001% (w/v) to 5% (w/v); and the pH is 6.0 or more.

(7) The pharmaceutical composition according to (3), (5) or (6), wherein the content of edetic acid or a salt thereof ranges from 0.005% (w/v) to 0.05% (w/v).

(8) The pharmaceutical composition according to any one of (4) to (7), wherein the content of the boric acid or a salt thereof ranges from 0.001% (w/v) to 1% (w/v).

(9) The pharmaceutical composition according to any one of (1) to (8), wherein the dorzolamide or a salt thereof is dorzolamide hydrochloride.

(10) The pharmaceutical composition according to any one of (1) to (9), wherein the brimonidine or a salt thereof is brimonidine tartrate.

(11) The pharmaceutical composition according to any one of (1) to (10), wherein the content of the dorzolamide or a salt thereof ranges from 0.1% (w/v) to 5% (w/v).

(12) The pharmaceutical composition according to (11), wherein the content of the dorzolamide or a salt thereof is 1% (w/v) or 2% (w/v).

(13) The pharmaceutical composition according to any one of (1) to (12), wherein the content of the brimonidine or a salt thereof ranges from 0.01% (w/v) to 2% (w/v).

(14) The pharmaceutical composition according to (13), wherein the content of the brimonidine or a salt thereof is 0.1% (w/v) or 0.15% (w/v).

(15) The pharmaceutical composition according to any one of (1) to (14), wherein the pH ranges from 6.0 to 8.0.

(16) The pharmaceutical composition according to any one of (1) to (15), which is used for treatment of glaucoma or ocular hypertension.

(17) The pharmaceutical composition according to any one of (1) to (16), which is placed in a multiple-dose container.

(18) A product, which is provided with the pharmaceutical composition according to any one of (1) to (17) and a multiple-dose container.

(19) Use of the pharmaceutical composition according to any one of (1) to (17) in the manufacture of a medicament for treatment of glaucoma or ocular hypertension.

(20) A method for improving preservative efficacy, which comprises formulating a pharmaceutical composition that comprises brimonidine or a salt thereof and does not comprise a preservative or comprises a preservative in a prescribed amount and has a pH of 6.0 or more, with dorzolamide or a salt thereof, wherein: the prescribed amount is such that the common logarithmic value of the ratio (B/A) of the number (B) of bacteria at the time of inoculation to the number (A) of the bacteria, when the number of the viable bacteria is measured after inoculating Escherichia Coli ATCC 8739 in a test sample that consists of the preservative and water and mixing homogeneously such that the concentration of the bacterial suspension is within the range of 10⁵-10⁶ cfu/mL, and then collecting 1 mL of the test sample using a micropipette after the test sample has been stored for seven days at 20° C.-25° C. while shielded from light, is 2.0 or less.

(21) A method for improving the preservative efficacy, which comprises formulating a pharmaceutical composition that comprises brimonidine or a salt thereof and has a pH of 6.0 or more, with dorzolamide or a salt thereof, wherein: the pharmaceutical composition before the formulation with the dorzolamide or a salt thereof is such that the common logarithmic value of the ratio (B/A) of the number (B) of bacteria at the time of inoculation to the number (A) of the bacteria, when the number of the viable bacteria is measured after inoculating Escherichia Coli ATCC 8739 in the pharmaceutical composition and mixing homogeneously such that the concentration of the bacterial suspension is within the range of 10⁵-10⁶ cfu/mL, and then collecting 1 mL of the pharmaceutical composition using a micropipette after the test sample has been stored for seven days at 20° C.-25° C. while shielded from light, is 2.0 or less.

Furthermore, any two or more configurations of (1) to (19) above can be selected and combined.

### Effects of the Invention

The present invention addresses the problem of providing a pharmaceutical composition that comprises dorzolamide or a salt thereof and brimonidine or a salt thereof and can exhibit a sufficient preservative effect in spite of comprising no preservative or comprising a low content of a preservative.

### PREFERRED MODE FOR CARRYING OUT THE INVENTION

Hereafter, the present invention will be described in detail. Dorzolamide comprised in the pharmaceutical composition of the present invention is a compound represented by a chemical name (4S, 6S)-4-Ethylamino-6-methyl-5,6-dihydro-4H-thieno[2,3-b]thiopyran-2-sulfonamide 7,7-dioxide.

Brimonidine comprised in the pharmaceutical composition of the present invention is a compound represented by a chemical name 5-Bromo-N-(4,5-dihydro-1H-imidazol-2-yl)quinoxaline-6-amine.

Dorzolamide and brimonidine comprised in the pharmaceutical composition of the present invention may be salts, and the salts are not particularly limited, as long as they are pharmaceutically acceptable salts. Examples of such a salt include a salt with inorganic acid, a salt with organic acid, a quaternary ammonium salt, a salt with halogen ion, a salt with alkali metal, a salt with alkaline-earth metal, a metal salt, and a salt with organic amine. Examples of a salt with inorganic acid include salts with hydrochloric acid, hydrobromic acid, hydroiodic acid, nitric acid, sulfuric acid, phosphoric acid, and the like. Examples of a salt with organic acid include salts with acetic acid, oxalic acid, fumaric acid, maleic acid, succinic acid, malic acid, citric acid, tartaric acid, adipic acid, gluconic acid, glucoheptonic acid, glucuronic acid, terephthalic acid, methansulfonic acid, alanine, lactic acid, hippuric acid, 1,2-ethanedisulfonic acid, isethionic acid, lactobionic acid, oleic acid, gallic acid, pamoic acid, polygalacturonic acid, stearic acid, tannic acid, trifluoromethanesulfonic acid, benzenesulfonic acid, p-toluenesulfonic acid, lauryl sulfate, methyl sulfate, naphthalenesulfonic acid, sulfosalicylic acid, and the like. Examples of a quaternary ammonium salt include salts with methyl bromide, methyl iodide, and the like. Examples of a salt with halogen ion include salts with chloride ion, bromide ion, iodide ion, and the like. Examples of a salt with alkali metal include salts with lithium, sodium, potassium, and the like. Examples of a salt with alkaline-earth metal include salts with calcium, magnesium, and the like. Examples of a metal salt include salts with iron, zinc, and the like. Examples of a salt with organic amine include salts with triethylenediamine, 2-aminoethanol, 2,2′-iminobis (ethanol), 1-deoxy-1-(methylamino)-2-D-sorbitol, 2-amino-2-(hydroxymethyl)-1,3-propanediol, procaine, N,N′-bis(phenylmethyl)-1,2-ethanediamine, and the like. As a salt of dorzolamide, monohydrochloride (dorzolamide hydrochloride) is particularly preferred. As a salt of brimonidine, monotartrate (brimonidine tartrate) is particularly preferred and mono-(2R,3R)-tartrate is the most preferred.

Dorzolamide, brimonidine and salts thereof to be comprised in the pharmaceutical composition of the present invention may be in the form of hydrate or solvation product.

The content of dorzolamide or a salt thereof to be comprised in the pharmaceutical composition of the present invention is not particularly limited, as long as the content is an amount sufficient for exhibition of desired drug effects and preservative efficacy, and ranges from preferably 0.1% (w/v) to 5% (w/v), more preferably 0.2% (w/v) to 3% (w/v), further preferably 0.5% (w/v) to 2% (w/v), and further more preferably 0.7% (w/v) to 1.2% (w/v), and is particularly preferably 1% (w/v). 1% (w/v) or 2% (w/v) is the most preferable. Note that when the pharmaceutical composition of the present invention comprises a salt of dorzolamide, these values are contents in terms of free dorzolamide. In addition, the term “% (w/v)” refers to the mass (g) of an object component (here, dorzolamide) comprised in 100 mL of the pharmaceutical composition of the present invention. The same applies to the followings unless otherwise specified.

The content of brimonidine or a salt thereof to be comprised in the pharmaceutical composition of the present invention is not particularly limited, as long as the content is an amount sufficient for exhibiting desired drug effects, and ranges from preferably 0.01% (w/v) to 2% (w/v), more preferably 0.02% (w/v) to 1% (w/v), further preferably 0.05% (w/v) to 0.5% (w/v), and particularly preferably 0.07% (w/v) to 0.2% (w/v), and is most preferably 0.1% (w/v) or 0.15% (w/v). Note that when the pharmaceutical composition of the present invention comprises a salt of brimonidine, these values are contents in terms of free brimonidine.

The content of dorzolamide or a salt thereof is, in view of therapeutic effects and preservative effects, preferably 1 to 30 times, more preferably 3 to 25 times, and further preferably 5 to 20 times more than the content of brimonidine or a salt thereof.

The pharmaceutical composition of the present invention exhibits a sufficient preservative effect, so that it can comprise no preservative or comprise a preservative in a prescribed amount. The term, “prescribed amount” refers to an amount such that the relevant preservative does not individually exhibit any preservative effect, and specifically may be an amount such that the common logarithmic value of the ratio (B/A) of the number (B) of bacteria at the time of inoculation to the number (A) of the bacteria, when the number of the viable bacteria is measured after inoculating Escherichia Coli ATCC 8739 in a test sample that consists of the preservative and water and mixing homogeneously such that the concentration of the bacterial suspension is within the range of 10⁵-10⁶ cfu/mL, and then collecting 1 mL of the test sample using a micropipette after the test sample has been stored for seven days at 20° C.-25° C. while shielded from light, is 2.0 or less, preferably 1.5 or less, more preferably 1.2 or less, further preferably 1.0 or less, particularly preferably 0.8 or less, and most preferably 0.7 or less. In particular, in the pharmaceutical composition of the present invention, the content of benzalkonium chloride as a preservative is desirably low or no such preservative is desirably comprised.

When the pharmaceutical composition of the present invention comprises benzalkonium chloride, the benzalkonium chloride is preferably comprised in a prescribed amount. The term, “prescribed amount” refers to an amount such that the relevant preservative does not individually exhibit any preservative effect, and specifically may be an amount such that the common logarithmic value of the ratio (B/A) of the number (B) of bacteria at the time of inoculation to the number (A) of the bacteria, when the number of the viable bacteria is measured after inoculating Escherichia Coli ATCC 8739 in a test sample that consists of benzalkonium chloride and water and mixing homogeneously such that the concentration of the bacterial suspension is within the range of 10⁵-10⁶ cfu/mL, and then collecting 1 mL of the test sample using a micropipette after the test sample has been stored for seven days at 20° C.-25° C. while shielded from light, is 2.0 or less, preferably 1.5 or less, more preferably 1.2 or less, further preferably 1.0 or less, particularly preferably 0.8 or less, and most preferably 0.7 or less. More specifically, the content of benzalkonium chloride is preferably 0.001% (w/v) or less, more preferably 0.0007% (w/v) or less, further preferably 0.0005% (w/v) or less, further more preferably 0.0003% (w/v) or less, particularly preferably 0.0001% (w/v) or less, and most preferably substantially no benzalkonium chloride is comprised.

Furthermore, the content of a quaternary ammonium salt to be used as a preservative other than benzalkonium chloride is desirably low or no quaternary ammonium salt is desirably comprised therein. Examples of a quaternary ammonium salt other than benzalkonium chloride include benzalkonium bromide, benzethonium chloride, and benzododecinium bromide. When the pharmaceutical composition of the present invention comprises a quaternary ammonium salt other than benzalkonium chloride, the quaternary ammonium salt is preferably comprised in a prescribed amount. Here, the term “prescribed amount” refers to, for example: an amount such that the relevant quaternary ammonium salt does not individually exhibit a preservative effect; and specifically may be an amount such that the common logarithmic value of the ratio (B/A) of the number (B) of bacteria at the time of inoculation to the number (A) of the bacteria, when the number of the viable bacteria is measured after inoculating Escherichia Coli ATCC 8739 in a test sample that consists of the preservative (a quaternary ammonium salt other than benzalkonium chloride) and water and mixing homogeneously such that the concentration of the bacterial suspension is within the range of 10⁵-10⁶ cfu/mL, and then collecting 1 mL of the test sample using a micropipette after the test sample has been stored for seven days at 20° C.-25° C. while shielded from light, is 2.0 or less, preferably 1.5 or less, more preferably 1.2 or less, further preferably 1.0 or less, particularly preferably 0.8 or less, and most preferably 0.7 or less. More specifically, the content of a quaternary ammonium salt other than benzalkonium chloride differs depending on the type, and the quaternary ammonium salt is not comprised, for example, preferably at 0.01% (w/v) or more (the content is less than 0.01% (w/v)), more preferably at 0.005% (w/v) or more, further preferably at 0.001% (w/v) or more, further more preferably at 0.0005% (w/v) or more, and particularly preferably at 0.0001% (w/v) or more. Substantially no quaternary ammonium salt comprised therein is the most preferable.

The pharmaceutical composition of the present invention desirably comprises a low content of a preservative other than quaternary ammonium salts or desirably does not comprise such a preservative. Examples of the preservative other than quaternary ammonium salts include sorbic acid, potassium sorbate, methyl parahydroxybenzoate, propyl parahydroxybenzoate, chlorobutanol, chlorhexidine, boric acid or a salt thereof, and edetic acid or a salt thereof. When the pharmaceutical composition of the present invention comprises the preservative other than quaternary ammonium salts, the preservative is preferably comprised in a prescribed amount. Here, the term “prescribed amount” refers to, for example: an amount such that the preservative individually exhibits preservative effects; and specifically may be an amount such that the common logarithmic value of the ratio (B/A) of the number (B) of bacteria at the time of inoculation to the number (A) of the bacteria, when the number of the viable bacteria is measured after inoculating Escherichia Coli ATCC 8739 in a test sample that consists of the preservative (a preservative other than quaternary ammonium salts) and water and mixing homogeneously such that the concentration of the bacterial suspension is within the range of 10⁵-10⁶ cfu/mL, and then collecting 1 mL of the test sample using a micropipette after the test sample has been stored for seven days at 20° C.-25° C. while shielded from light, is 2.0 or less, preferably 1.5 or less, more preferably 1.2 or less, further preferably 1.0 or less, particularly preferably 0.8 or less, and most preferably 0.7 or less. More specifically, the content of a preservative (particularly, boric acid or a salt thereof) other than quaternary ammonium salts differs depending on the type, and the preservative is comprised preferably at 5% (w/v) or less, more preferably at 3% (w/v) or less, and further preferably at 1% (w/v) or less, and is not comprised further more preferably at 0.5% (w/v) or more (the content is less than 0.5% (w/v)), is not comprised particularly preferably at 0.10% (w/v) or more (the content is less than 0.10%(w/v)), is not comprised further preferably at 0.05% (w/v) or more, is not comprised further more preferably at 0.01% (w/v) or more, is not comprised even more preferably at 0.005% (w/v) or more, is not comprised particularly preferably at 0.001% (w/v) or more. Most preferably substantially no preservatives other than quaternary ammonium salts is comprised therein. Examples of a salt of boric acid include borax, sodium borate, and potassium borate. Note that when the pharmaceutical composition of the present invention comprises a salt of boric acid, these values are contents in terms of free boric acid. Moreover, edetic acid or a salt thereof is often added as a stabilizing agent to a pharmaceutical composition and known to have a preservative effect. Accordingly, when the pharmaceutical composition of the present invention comprises edetic acid or a salt thereof, the content thereof as a total content is higher than 0% (w/v) (e.g., 0.0001% or more, 0.0005% or more, 0.001% or more, 0.002% or more, 0.003% or more, 0.005% or more, and 0.007% or more), is preferably 2% (w/v) or less, more preferably 1% (w/v) or less, further preferably 0.5% (w/v) or less, further more preferably 0.3% (w/v) or less, even more preferably 0.1% (w/v) or less, particularly preferably 0.07% (w/v) or less, and is most preferably 0.05% (w/v) or less. Examples of a salt of edetic acid include monosodium edetate, disodium edetate, tetrasodium edetate, and sodium citrate. Disodium edetate is preferred, and disodium edetate dihydrate is particularly preferred. In particular, the pharmaceutical composition of the present invention preferably does not comprise a preservative other than boric acid and a salt thereof, as well as edetic acid and a salt thereof. Note that when the pharmaceutical composition of the present invention comprises a salt of edetic acid or a hydrate thereof, these values are contents calculated based on the mass of such a salt of edetic acid or a hydrate thereof. The term “preservative” in the present invention refers to a component denoted as a preservative in the pharmaceutical composition, and examples thereof do not include a component such as dorzolamide or a salt thereof in the pharmaceutical composition of the present invention, which exhibits a preservative effect, but is not indicated as a preservative.

Additives can be used as necessary for the pharmaceutical composition of the present invention and examples of additives that can be added herein include a surfactant, a buffering agent, a tonicity agent, a stabilizing agent, an antioxidant, and a high molecular weight polymer.

The pharmaceutical composition of the present invention can be formulated with a surfactant that can be used as an additive for a pharmaceutical product, such as a cationic surfactant, an anionic surfactant, or a nonionic surfactant. Examples of an anionic surfactant include a phospholipid, and an example of a phospholipid is lecithin. Examples of a cationic surfactant include an alkyl amine salt, an alkylaminepolyoxyethylene adduct, a fatty acid triethanolamine monoester salt, an acylaminoethyldiethyl amine salt, a fatty acid-polyamine condensate, an alkyl imidazoline, a 1-acylaminoethyl-2-alkylimidazoline, and a 1-hydroxylethyl-2-alkylimidazoline. Examples of a nonionic surfactant include a polyoxyethylene fatty acid ester, a polyoxyethylene sorbitan fatty acid ester, a polyoxyethylene hardened castor oil, a polyoxyethylene castor oil, polyoxyethylene polyoxypropylene glycol, a sucrose fatty acid ester, and vitamin E TPGS.

An example of a polyoxyethylene fatty acid ester is polyoxyl 40 stearate.

Examples of a polyoxyethylene sorbitan fatty acid ester include polysorbate 80, polysorbate 60, polysorbate 40, polyoxyethylene sorbitan monolaurate, polyoxyethylene sorbitan trioleate, and polysorbate 65.

As polyoxyethylene hardened castor oil, various types of polyoxyethylene hardened castor oil with different polymerization numbers of ethylene oxide can be used, and the polymerization number of ethylene oxide ranges from preferably 10 to 100, more preferably 20 to 80, particularly preferably 40 to 70, and is most preferably 60. Specific examples of polyoxyethylene hardened castor oil include polyoxyethylene hardened castor oil 10, polyoxyethylene hardened castor oil 40, polyoxyethylene hardened castor oil 50, and polyoxyethylene hardened castor oil 60.

As polyoxyethylene castor oil, various types of polyoxyethylene castor oil with different polymerization numbers of ethylene oxide can be used, and the polymerization number of ethylene oxide ranges from preferably 5 to 100, more preferably 20 to 50, particularly preferably 30 to 40, and is most preferably 35. Specific examples of polyoxyethylene castor oil include polyoxyl 5 castor oil, polyoxyl 9 castor oil, polyoxyl 15 castor oil, polyoxyl 35 castor oil, and polyoxyl 40 castor oil.

Examples of polyoxyethylene polyoxypropylene glycol include polyoxyethylene (160) polyoxypropylene (30) glycol, polyoxyethylene (42) polyoxypropylene (67) glycol, polyoxyethylene (54) polyoxypropylene (39) glycol, polyoxyethylene (196) polyoxypropylene (67) glycol, and polyoxyethylene (20) polyoxypropylene (20) glycol.

An example of a sucrose fatty acid ester is a sucrose stearic acid ester.

Vitamin E TPGS may also be referred to as tocopherol polyethylene glycol 1000 succinate.

When the pharmaceutical composition comprising a preservative used therein of the present invention is formulated with a surfactant, the content of the surfactant can be adjusted as appropriate by varying the type or the like of the surfactant and ranges from preferably 0.001% (w/v) to 10% (w/v), more preferably 0.01% (w/v) to 5%(w/v), and further preferably 0.1% (w/v) to 3% (w/v), and most preferably 0.2% (w/v) to 2% (w/v).

The pharmaceutical composition of the present invention can be formulated with a buffering agent that can be used as an additive for pharmaceutical products. Examples of such a buffering agent include phosphoric acid or a salt thereof, citric acid or a salt thereof, acetic acid or a salt thereof, carbonic acid or a salt thereof, tartaric acid or a salt thereof, ε-aminocaproic acid, and trometamol. Examples of phosphate include sodium phosphate, sodium dihydrogenphosphate, disodium hydrogenphosphate, potassium phosphate, potassium dihydrogenphosphate, and dipotassium hydrogenphosphate. Examples of citrate include sodium citrate and disodium citrate. Examples of acetate include sodium acetate and potassium acetate. Examples of carbonate include sodium carbonate and sodium hydrogencarbonate. Examples of tartrate include sodium tartrate and potassium tartrate. Citric acid or a salt thereof is preferred and sodium citrate is particularly preferred. When the pharmaceutical composition of the present invention is formulated with a buffering agent, the content of the buffering agent can be adjusted as appropriate by varying the type or the like of the buffering agent, and ranges from preferably 0.001% (w/v) to 10% (w/v), more preferably 0.01% (w/v) to 5% (w/v), and further preferably 0.1% (w/v) to 3% (w/v), and is most preferably 0.2% (w/v) to 2% (w/v).

The pharmaceutical composition of the present invention can be formulated as appropriate with a tonicity agent that can be used as an additive for pharmaceutical products. Examples of the tonicity agent include an ionic tonicity agent and a nonionic tonicity agent. Examples of the ionic tonicity agent include sodium chloride, potassium chloride, calcium chloride, and magnesium chloride, and sodium chloride is preferred. Examples of the nonionic tonicity agent include glycerin, propylene glycol, sorbitol, and mannitol, and mannitol is preferred. When the pharmaceutical composition of the present invention is formulated with the tonicity agent, the content of the tonicity agent can be adjusted as appropriate by varying the type or the like of the tonicity agent, and ranges from preferably 0.01% (w/v) to 10% (w/v), more preferably 0.02% (w/v) to 7% (w/v), further preferably 0.1% (w/v) to 5% (w/v), particularly preferably 0.5% (w/v) to 4% (w/v), and most preferably 0.8% (w/v) to 3% (w/v).

The pharmaceutical composition of the present invention can be formulated as appropriate with a stabilizing agent that can be used as an additive for pharmaceutical products. Examples of the stabilizing agent include edetic acid, monosodium edetate, disodium edetate, tetrasodium edetate, and sodium citrate. Disodium edetate is preferred and disodium edetate dihydrate is particularly preferred. When the pharmaceutical composition of the present invention is formulated with the stabilizing agent, the content of the stabilizing agent can be adjusted as appropriate by varying the type or the like of the stabilizing agent, and ranges from preferably 0.0001% (w/v) to 2% (w/v), more preferably 0.0005% (w/v) to 1% (w/v), further preferably 0.001% (w/v) to 0.5% (w/v), further more preferably 0.002% (w/v) to 0.3% (w/v), even more preferably 0.003% (w/v) to 0.1% (w/v), particularly preferably 0.005% (w/v) to 0.07% (w/v), and most preferably 0.007% (w/v) to 0.05% (w/v).

The pharmaceutical composition of the present invention can be formulated as appropriate with an antioxidant that can be used as an additive for pharmaceutical products. Examples of the antioxidant include ascorbic acid, tocopherol, dibutylhydroxytoluene, butylated hydroxyanisole, sodium erythorbate, propyl gallate, and sodium sulfite. When the pharmaceutical composition of the present invention is formulated with the antioxidant, the content of the antioxidant can be adjusted as appropriate by varying the type or the like of the antioxidant, and ranges from preferably 0.0001% (w/v) to 1% (w/v), more preferably 0.0005% (w/v) to 0.1% (w/v), further preferably 0.001% (w/v) to 0.02% (w/v), and most preferably 0.005% (w/v) to 0.010% (w/v).

The pharmaceutical composition of the present invention can be formulated as appropriate with a high molecular weight polymer that can be used as an additive for pharmaceutical products. Examples of the high molecular weight polymer include methyl cellulose, ethyl cellulose, hydroxymethyl cellulose, hydroxyethyl cellulose, hydroxypropyl cellulose, hydroxyethylmethyl cellulose, hydroxypropylmethyl cellulose, carboxymethyl cellulose, sodium carboxymethyl cellulose, hydroxypropylmethyl cellulose acetate succinate, hydroxypropylmethylcellulose phthalate, carboxymethylethyl cellulose, cellulose acetate phthalate, polyvinyl pyrrolidone, polyvinyl alcohol, carboxyvinyl polymer, and polyethylene glycol, and hydroxyethyl cellulose is preferred. When the pharmaceutical composition of the present invention is formulated with the high molecular weight polymer, the content of the high molecular weight polymer can be adjusted as appropriate by varying the type or the like of the high molecular weight polymer, and ranges from preferably 0.001% (w/v) to 5% (w/v), more preferably 0.01% (w/v) to 3% (w/v), further preferably 0.1% (w/v) to 2% (w/v), and most preferably 0.2% (w/v) to 1% (w/v).

The pharmaceutical composition of the present invention can be formulated as appropriate with a pH adjuster that can be used as an additive for pharmaceutical products. Examples of the pH adjuster include hydrochloric acid, phosphoric acid, citric acid, acetic acid, sodium hydroxide, potassium hydroxide, sodium carbonate, and sodium hydrogencarbonate.

The pharmaceutical composition of the present invention particularly preferably comprises a combination of hydroxyethyl cellulose as a high molecular weight polymer, mannitol as a tonicity agent, and citric acid or a salt thereof as a buffering agent. With this combination, the pharmaceutical composition of the present invention can achieve rapid sterilization. In this case, the content of each component when the pharmaceutical composition of the present invention is formulated with these components is as follows: preferably, the content of hydroxyethyl cellulose ranges from 0.001% (w/v) to 5% (w/v), the content of mannitol ranges from 0.01% (w/v) to 10% (w/v), and the content of citric acid or a salt thereof ranges from 0.001% (w/v) to 10% (w/v); more preferably, the content of hydroxyethyl cellulose ranges from 0.01% (w/v) to 3% (w/v), the content of mannitol ranges from 0.02% (w/v) to 7% (w/v), and the content of citric acid or a salt thereof ranges from 0.01% (w/v) to 5% (w/v); further preferably the content of hydroxyethyl cellulose ranges from 0.1% (w/v) to 2% (w/v), the content of mannitol ranges from 0.1% (w/v) to 5% (w/v), and the content of citric acid or a salt thereof ranges from 0.1% (w/v) to 3% (w/v); and most preferably the content of hydroxyethyl cellulose ranges from 0.2% (w/v) to 1% (w/v), the content of mannitol ranges from 0.8% (w/v) to 3% (w/v), and the content of citric acid or a salt thereof ranges from 0.2% (w/v) to 2% (w/v).

The pH of the pharmaceutical composition of the present invention is 6.0 or more, and the upper limit of the pH is, in view of solubility and stability of dorzolamide and brimonidine, preferably 8.0, more preferably 7.5, and further preferably 7.0, and is most preferably 6.8. The pH ranges from preferably 6.0 to 8.0, more preferably 6.0 to 7.5, further preferably 6.0 to 7.0, and most preferably 6.0 to 6.8.

The pharmaceutical composition of the present invention can be placed in a unit-dose container or a multiple-dose container, and is preferably placed in a multiple-dose container. The unit-dose container is a single-use container, and the multiple-dose container is designed so that a cap and the like can be freely opened and closed for multiple uses. The pharmaceutical composition can also be placed in a PFMD (Preservative Free Multi Dose) container having a special structure for exhibiting preservative effects such as a backflow prevention function. The material of such a container is not particularly limited, and for example, a container made of polyethylene (PE), polypropylene (PP), polyethylene terephthalate (PET), or the like can be used.

The dosage form of the pharmaceutical composition of the present invention is not particularly limited, as long as it can be used as a pharmaceutical product. Particularly, eye drops are preferred and can be produced according to a general method in the art.

The pharmaceutical composition of the present invention is useful as a therapeutic agent for glaucoma or ocular hypertension.

When the pharmaceutical composition of the present invention is administered, uses and dosage thereof are not particularly limited, as long as they are sufficient for exhibiting desired drug effects. One to three drops 1 to 3 times/day is preferred, one to two drops 1 to 2 times/day is more preferred, and one drop 2 times/day is the most preferred.

The pharmaceutical composition of the present invention is useful for contact lenses (wearer). Types of contact lenses to be applied are not particularly limited, and specific examples thereof include hard contact lenses and soft contact lenses. Gas-permeable contact lenses may also be used herein. Examples of soft contact lenses include hydrous soft contact lenses, non-hydrous soft contact lenses, and (nonionic) silicone hydrogel soft contact lenses.

The above detailed description of the pharmaceutical composition of the present invention is also applied to products provided with the pharmaceutical composition of the present invention and a multiple-dose container (comprising the pharmaceutical composition), use of the pharmaceutical composition of the present invention in the manufacture of a medicament for treatment of glaucoma or ocular hypertension, and a method for improving preservative efficacy.

A method for improving the preservative efficacy of the present invention is preferably a method for improving the preservative efficacy by formulating the pharmaceutical composition (particularly, a pharmaceutical composition having a pH of 6.0 or more) that comprises brimonidine or a salt thereof and is placed in a multiple-dose container, with dorzolamide or a salt thereof.

The method for improving preservative efficacy of the present invention is preferably a method for improving preservative efficacy, which comprises formulating a pharmaceutical composition that does not comprise a preservative or comprises a preservative in a prescribed amount, has a pH of 6.0 or more, and is placed in a multiple-dose container, with dorzolamide or a salt thereof. Here, the terms “preservative” and “prescribed amount” in the method for improving preservative efficacy of the present invention may be similar to those described above for the above preservatives in the present invention and the pharmaceutical composition of the present invention. In particular, the method for improving preservative efficacy of the present invention is preferably a method for improving preservative efficacy, which comprises formulating a pharmaceutical composition that does not comprise benzalkonium chloride, does not comprise boric acid or a salt thereof or comprises boric acid or a salt thereof in an amount of less than 0.001% (w/v), has a pH of 6.0 or more, and is placed in a multiple-dose container, with dorzolamide or a salt thereof.

In the method for improving preservative efficacy of the present invention, a pharmaceutical composition before formulation of the same with dorzolamide or a salt thereof is such that the common logarithmic value of the ratio (B/A) of the number (B) of bacteria at the time of inoculation to the number (A) of the bacteria, when the number of the viable bacteria is measured after inoculating Escherichia Coli ATCC 8739 in the pharmaceutical composition, mixing homogeneously such that the concentration of the bacterial suspension is within the range of 10⁵-10⁶ cfu/mL, and then collecting 1 mL of the pharmaceutical composition using a micropipette after the pharmaceutical composition has been stored for seven days at 20° C.-25° C. while shielded from light, is preferably 2.0 or less, more preferably 1.5 or less, and further preferably 1.0 or less.

In the method for improving preservative efficacy of the present invention, the pharmaceutical composition after formulation of the same with dorzolamide or a salt thereof is such that the common logarithmic value of the ratio (B/A) of the number (B) of bacteria at the time of inoculation to the number (A) of the bacteria, when the number of the viable bacteria is measured after inoculating Escherichia Coli ATCC 8739 in the pharmaceutical composition, mixing homogeneously such that the concentration of the bacterial suspension is within the range of 10⁵-10⁶ cfu/mL, and then collecting 1 mL of the pharmaceutical composition using a micropipette after the pharmaceutical composition has been stored for seven days at 20° C.-25° C. while shielded from light, is preferably 2.5 or more, more preferably 3.0 or more, further preferably 3.5 or more, and even more preferably 4.0 or more. Alternatively, in the method for improving preservative efficacy of the present invention, the above pharmaceutical composition after formulation of the same with dorzolamide or a salt thereof preferably meets the criteria “Category IA” according to “antimicrobial preservative effectiveness test” in The Japanese Pharmacopoeia 16th Edition (JP16) General Information.

### EXAMPLES

Formulation examples and preservative efficacy test results are shown below for better understanding of the present invention, but are not intended to limit the scope of the present invention.

### Formulation Examples

Typical formulation examples of the present invention are shown below. Note that the amount of each component formulated in the following formulation examples is the content in 1 mL of the relevant formulation.

### Formulation Example 1 (In Multiple-Dose Container)

Dorzolamide 10 mg
Brimonidine 5 mg
Diluted hydrochloric acid Appropriate amount
Sodium hydroxide Appropriate amount
Purified water Appropriate amount

### Formulation Example 2 (In Multiple-Dose Container)

Dorzolamide 20 mg
Brimonidine 5 mg
Diluted hydrochloric acid Appropriate amount
Sodium hydroxide Appropriate amount
Purified water Appropriate amount

In addition, desired compositions can be obtained by adequately adjusting the types and the amounts of dorzolamide, brimonidine and additives in the above formulation examples 1 and 2.

### Preservative Efficacy Test (1)

### 1. Preparation of Test Formulation

Dorzolamide hydrochloride (1 g), brimonidine tartrate (0.1 g), sodium citrate hydrate (0.294 g), boric acid (0.7 g), D-mannitol (2.0 g), and disodium edetate dihydrate (0.05 g) were dissolved in water, and then subjected to filter sterilization. The resulting solution was adjusted using a pH regulator to have pH 6.5, and then water was added to the solution such that the total amount was 100 mL, so that the formulation of example 1 was prepared. A formulation of comparative example 1 was prepared by the preparation method same as that in example 1 except for changing the pH.

### 2. Test Method

Following strains were used as test microorganisms. Bacteria: Escherichia Coli ATCC 8739 (may also be referred to as E. coli) Pseudomonas aeruginosa ATCC 9027 (may also be referred to as P. aeruginosa) Staphylococcus aureus ATCC 6538 (may also be referred to as S. aureus)
Yeasts and fungi: Candida albicans ATCC 10231 (may also be referred to as C. albicans) Aspergillus brasiliensis ATCC16404 (may also be referred to as A. brasiliensis)

An inoculation microbial suspension was inoculated in a test sample so that the concentration of the microbial suspension in the test sample comprising each formulation ranged from 10⁵ to 10⁶ cells/mL (all 5 strains). Specifically, the inoculation microbial suspension was prepared to have a concentration of 10⁷-10⁸ cfu/mL. The inoculation microbial suspension was then inoculated in each test sample comprising the formulation of example 1 or comparative example 1 to have a concentration of 10⁵-10⁶ cfu/mL, and then the resultant was mixed homogeneously, thereby obtaining a sample. These samples were stored at 20° C. to 25° C. while shielded from light, 1 mL of each sample was collected at each sampling point (7 days later, 14 days later or 28 days later) using a micropipette, and then the number of viable microorganisms was measured. At each sampling point, the lid for each sample solution was opened, sampling was performed, and then the lid was closed.

### 3. Test results and discussion

Test results are shown in table 1. The test results in table 1 are the common logarithmic value of the ratio (B/A) of the number (B) of microorganisms at the time of inoculation to the number (A) of the same when the number of the viable microorganisms is measured. For example, the test result of “1” indicates that the measured number of viable microorganisms decreased to 10% of the number of inoculated microorganisms. Furthermore, the formulations were determined whether or not they meet the criteria “Category IA” according to the “antimicrobial preservative effectiveness test” in The Japanese Pharmacopoeia 16th Edition (JP16) General Information.

TABLE 1

| | | Comparative |
|---|---|---|
| Component % (w/v) | Example 1 | Example 1 |
| Dorzolamide hydrochloride | 1.0 | 1.0 |
| Brimonidine tartrate | 0.1 | 0.1 |
| Sodium citrate hydrate | 0.294 | 0.294 |
| Boric acid | 0.7 | 0.7 |
| Disodium edetate dihydrate | 0.05 | 0.05 |
| D-mannitol | 2.0 | 2.0 |
| Diluted hydrochloric acid | Appropriate | Appropriate |
| | amount | amount |
| Sodium hydroxide | Appropriate | Appropriate |
| | amount | amount |
| Purified water | Appropriate | Appropriate |
| | amount | amount |
| pH | 6.5 | 5.8 |
| Preservative efficacy test | | |

| | | |
|---|---|---|
| Strain | Sampling | Result (log reduction value) |

| | | | | |
|---|---|---|---|---|
| E. coli | 7 | d | 3.3 | 1.0 |
| | 14 | d | >4.7 | 2.4 |
| | 28 | d | >4.7 | >4.7 |
| P. aeruginosa | 7 | d | >4.7 | >4.7 |
| | 14 | d | >4.7 | >4.7 |
| | 28 | d | >4.7 | >4.7 |
| S. aureus | 7 | d | 2.5 | 2.7 |
| | 14 | d | >4.6 | >4.6 |
| | 28 | d | >4.6 | >4.6 |
| C. albicans | 7 | d | 0.2 | 0.0 |
| | 14 | d | 0.5 | 0.3 |
| | 28 | d | 2.3 | 1.5 |
| A. brasiliensis | 7 | d | 3.5 | 3.9 |
| | 14 | d | >4.2 | >4.2 |
| | 28 | d | >4.2 | >4.2 |

| | | |
|---|---|---|
| Determination | Conformity | Nonconformity |

As shown in table 1, of the formulations comprising dorzolamide hydrochloride and brimonidine tartrate, the formulation of example 1 having a pH of 6.5 exhibited strong preservative effect on all strains, and found to meet (conformity) the criteria “Category IA” according to the “antimicrobial preservative effectiveness test” in The Japanese Pharmacopoeia 16th Edition (JP16) General Information, whereas the formulation of comparative example 1 having a pH of 5.8 did not meet (nonconformity) the criteria.

1. A pharmaceutical composition, which comprises dorzolamide or a salt thereof and brimonidine or a salt thereof, and does not comprise a preservative or comprises a preservative in a prescribed amount, wherein:
the prescribed amount is such that the common logarithmic value of the ratio (B/A) of the number (B) of bacteria at the time of inoculation to the number (A) of the bacteria, when the number of the viable bacteria is measured after inoculating Escherichia Coli ATCC 8739 in a test sample that consists of the preservative and water and mixing homogeneously such that the concentration of the bacterial suspension is within the range of 10⁵-10⁶ cfu/mL, and then collecting 1 mL of the test sample using a micropipette after the test sample has been stored for seven days at 20° C.-25° C. while shielded from light, is 2.0 or less; and
the pH is 6.0 or more.

2. A pharmaceutical composition, which comprises dorzolamide or a salt thereof and brimonidine or a salt thereof, does not comprise benzalkonium chloride, and does not comprise a preservative other than benzalkonium chloride or comprises the same in a prescribed amount, wherein:
the prescribed amount is such that the common logarithmic value of the ratio (B/A) of the number (B) of bacteria at the time of inoculation to the number (A) of the bacteria, when the number of the viable bacteria is measured after inoculating Escherichia Coli ATCC 8739 in a test sample that consists of the preservative and water and mixing homogeneously such that the concentration of the bacterial suspension is within the range of 10⁵-10⁶ cfu/mL, and then collecting 1 mL of the test sample using a micropipette after the test sample has been stored for seven days at 20° C.-25° C. while shielded from light, is 2.0 or less; and
the pH is 6.0 or more.

3. The pharmaceutical composition according to claim 1, which comprises edetic acid or a salt thereof, wherein the content of the edetic acid or a salt thereof ranges from 0.0001% (w/v) to 2% (w/v).

4. The pharmaceutical composition according to claim 1, which comprises boric acid or a salt thereof, wherein the content of the boric acid or a salt thereof ranges from 0.0001% (w/v) to 5% (w/v).

5. A pharmaceutical composition, which comprises dorzolamide or a salt thereof and brimonidine or a salt thereof, does not comprise a preservative other than edetic acid or a salt thereof and boric acid or a salt thereof, or comprises the same in a prescribed amount, wherein:
the prescribed amount is such that the common logarithmic value of the ratio (B/A) of the number (B) of bacteria at the time of inoculation to the number (A) of the bacteria, when the number of the viable bacteria is measured after inoculating Escherichia Coli ATCC 8739 in a test sample that consists of the preservative and water and mixing homogeneously such that the concentration of the bacterial suspension is within the range of 10⁵-10⁶ cfu/mL, and then collecting 1 mL of the test sample using a micropipette after the test sample has been stored for seven days at 20° C.-25° C. while shielded from light, is 2.0 or less;
the content of the edetic acid or a salt thereof ranges from 0.0001% (w/v) to 2% (w/v);
the content of the boric acid or a salt thereof ranges from 0.0001% (w/v) to 5% (w/v); and
the pH is 6.0 or more.

6. A pharmaceutical composition, which comprises dorzolamide or a salt thereof and brimonidine or a salt thereof, does not comprise a preservative other than edetic acid or a salt thereof and boric acid or a salt thereof, wherein:
the content of the edetic acid or a salt thereof ranges from 0.0001% (w/v) to 2% (w/v);
the content of the boric acid or a salt thereof ranges from 0.0001% (w/v) to 5% (w/v); and
the pH is 6.0 or more.

7. The pharmaceutical composition according to claim 3, wherein the content of edetic acid or a salt thereof ranges from 0.005% (w/v) to 0.05% (w/v).

8. The pharmaceutical composition according to claim 4, wherein the content of the boric acid or a salt thereof ranges from 0.001% (w/v) to 1% (w/v).

9. The pharmaceutical composition according to claim 1, wherein the dorzolamide or a salt thereof is dorzolamide hydrochloride.

10. The pharmaceutical composition according to claim 1, wherein the brimonidine or a salt thereof is brimonidine tartrate.

11. The pharmaceutical composition according to claim 1, wherein the content of the dorzolamide or a salt thereof ranges from 0.1% (w/v) to 5% (w/v).

12. The pharmaceutical composition according to claim 11, wherein the content of the dorzolamide or a salt thereof is 1% (w/v) or 2% (w/v).

13. The pharmaceutical composition according to claim 1, wherein the content of the brimonidine or a salt thereof ranges from 0.01% (w/v) to 2% (w/v).

14. The pharmaceutical composition according to claim 13, wherein the content of the brimonidine or a salt thereof is 0.1% (w/v) or 0.15% (w/v).

15. The pharmaceutical composition according to claim 1, wherein the pH ranges from 6.0 to 8.0.

16. The pharmaceutical composition according to claim 1, which is used for treatment of glaucoma or ocular hypertension.

17. The pharmaceutical composition according to claim 1, which is placed in a multiple-dose container.

18. A product, which is provided with the pharmaceutical composition according to claim 1 and a multiple-dose container.

19. Use of the pharmaceutical composition according to claim 1 in the manufacture of a medicament for treatment of glaucoma or ocular hypertension.

20. A method for improving preservative efficacy, which comprises formulating a pharmaceutical composition that comprises brimonidine or a salt thereof and does not comprise a preservative or comprises a preservative in a prescribed amount and has a pH of 6.0 or more, with dorzolamide or a salt thereof, wherein:
the prescribed amount is such that the common logarithmic value of the ratio (B/A) of the number (B) of bacteria at the time of inoculation to the number (A) of the bacteria, when the number of the viable bacteria is measured after inoculating Escherichia Coli ATCC 8739 in a test sample that consists of the preservative and water and mixing homogeneously such that the concentration of the bacterial suspension is within the range of 10⁵-10⁶ cfu/mL, and then collecting 1 mL of the test sample using a micropipette after the test sample has been stored for seven days at 20° C.-25° C. while shielded from light, is 2.0 or less.

21. A method for improving preservative efficacy, which comprises formulating a pharmaceutical composition that comprises brimonidine or a salt thereof and has a pH of 6.0 or more, with dorzolamide or a salt thereof, wherein:
the pharmaceutical composition before the formulation with the dorzolamide or a salt thereof is such that the common logarithmic value of the ratio (B/A) of the number (B) of bacteria at the time of inoculation to the number (A) of the bacteria, when the number of the viable bacteria is measured after inoculating Escherichia Coli ATCC 8739 in the pharmaceutical composition and mixing homogeneously such that the concentration of the bacterial suspension is within the range of 10⁵-10⁶ cfu/mL, and then collecting 1 mL of the pharmaceutical composition using a micropipette after the test sample has been stored for seven days at 20° C.-25° C. while shielded from light, is 2.0 or less.
